(19) United States
(12) Patent Application Publication
(10) Pub. No.: US 2011/0207722 A1
(43) Pub. Date: Aug. 25, 2011

(54) TREATMENT OF ANXIETY DISORDERS

(75) Inventors: Dan Peters, Malmo (SE); John Paul Redrobe, Rodovre (DK); Alexander Norup Nielsen, Rungsted (DK)

(73) Assignee: NEUROSEARCH A/S, Ballerup (DK)

(21) Appl. No.: 13/059,807

(22) PCT Filed: Aug. 19, 2009
(86) PCT No.: PCT/EP2009/060704

§ 371 Date: May 9, 2011

(30) Foreign Application Priority Data
Aug. 21, 2008 (DK) PA 2008 01133

(51) Int. Cl. A61K 31/5513; A61K 31/46; A61P 25/22; A61P 25/00

(52) U.S. Cl. 514/221; 514/304

(57) ABSTRACT

This invention relates to the treatment of anxiety disorders. The invention furthermore relates to novel pharmaceutical compositions comprising a therapeutically effective amount of the compound exo-7-(8-H-8-aza-bicyclo[3.2.1]oct-3-yloxy)-chromen-2-one, or a therapeutically effective amount of the compound exo- 7-(8-H-8- aza-bicyclo[3.2.1]oct-3-yloxy)-chromen-2-one and a benzodiazepine drug.

### TECHNICAL FIELD

This invention relates to the treatment of anxiety disorders. The invention furthermore relates to novel pharmaceutical compositions comprising a therapeutically effective amount of the compound exo-7-(8-H-8-aza-bicyclo[3.2.1]oct-3-yloxy)-chromen-2-one, or a therapeutically effective amount of the compound exo-7-(8-H-8-aza-bicyclo[3.2.1]oct-3-yloxy)-chromen-2-one and a benzodiazepine drug.

### BACKGROUND ART

Anxiety disorders represent a significant public health issue and place a substantial economic burden on society. A number of drugs have either been developed or are being developed for treating anxiety disorders. Among the preferred medicines are the benzodiazepine drugs. These drugs are effective in quickly relieving the symptoms of anxiety. However, the body rapidly becomes tolerant to the therapeutic effects of these drugs and doses needed to induce benefit often need to be increased, leading to unwanted side effects. Importantly, long term treatment with these drugs often leads to dependence. As a result, benzodiazepine drugs are good for short-term help, but should not generally be used for longer periods. Thus there is a continued need for compounds or pharmaceutical compositions with an optimised pharmacological profile. Furthermore, there is a strong need to find effective compounds or pharmaceutical compositions without unwanted side effects associated with the drugs presently available.

WO 2006/035034 (NeuroSearch A/S) discloses novel chromen-2-one derivatives and their use as monoamine neurotransmitter re-uptake inhibitors. One of the compounds disclosed is exo-7-(8-H-8-aza-bicyclo[3.2.1]oct-3-yloxy)-chromen-2-one.

### SUMMARY OF THE INVENTION

It has surprisingly been shown that the compound exo-7-(8-H-8-aza-bicyclo[3.2.1]oct-3-yloxy)-chromen-2-one can be used for treating anxiety disorders.

In its first aspect the invention provides a method for treating an anxiety disorder comprising administering to a human a composition comprising the compound exo-7-(8-H-8-aza-bicyclo[3.2.1]oct-3-yloxy)-chromen-2-one, or a pharmaceutically acceptable salt thereof in a therapeutically-effective amount.

In another aspect the invention relates to pharmaceutical composition comprising a therapeutically effective amount of

(i) the compound exo-7-(8-H-8-aza-bicyclo[3.2.1]oct-3-yloxy)-chromen-2-one, or a pharmaceutically acceptable salt thereof; and

(ii) a benzodiazepine drug; or a pharmaceutically acceptable salt thereof; together with one or more adjuvants, excipients, carriers and/or diluents.

Other objects of the invention will be apparent to the person skilled in the art from the following detailed description and examples.

### DETAILED DISCLOSURE OF THE INVENTION

In its first aspect the invention provides a method for treating an anxiety disorder comprising administering to a human a composition comprising the compound exo-7-(8-H-8-aza-bicyclo[3.2.1]oct-3-yloxy)-chromen-2-one, or a pharmaceutically acceptable salt thereof in a therapeutically-effective amount.

In a second aspect the invention relates to a pharmaceutical composition for the treatment of an anxiety disorder in a human, said composition comprising a therapeutically-effective amount of the compound exo-7-(8-H-8-aza-bicyclo[3.2.1]oct-3-yloxy)-chromen-2-one, or a pharmaceutically acceptable salt thereof in admixture with one or more pharmaceuticaly acceptable adjuvants, excipients, carriers and/or diluents.

In a further aspect the invention relates to the use of the compound exo-7-(8-H-8-aza-bicyclo[3.2.1]oct-3-yloxy)-chromen-2-one, or a pharmaceutically acceptable salt thereof in a therapeutically-effective amount for the preparation of a medicament for the treatment of an anxiety disorder.

In a still further aspect the invention provides a pharmaceutical composition comprising a therapeutically effective amount of

(i) the compound exo-7-(8-H-8-aza-bicyclo[3.2.1]oct-3-yloxy)-chromen-2-one, or a pharmaceutically acceptable salt thereof; and

(ii) a benzodiazepine drug; or a pharmaceutically acceptable salt thereof; together with one or more adjuvants, excipients, carriers and/or diluents.

In an even further aspect the invention relates to a combination of

(i) the compound exo-7-(8-H-8-aza-bicyclo[3.2.1]oct-3-yloxy)-chromen-2-one, or a pharmaceutically acceptable salt thereof; and

(ii) a benzodiazepine drug; or a pharmaceutically acceptable salt thereof; for the manufacture of a medicament for the treatment, prevention or alleviation of an anxiety disorder.

In a still further aspect the invention relates to a kit of parts comprising at least two separate unit dosage forms (A) and (B):

(A) the compound exo-7-(8-H-8-aza-bicyclo[3.2.1]oct-3-yloxy)-chromen-2-one, or a pharmaceutically acceptable salt thereof; and

(B) a benzodiazepine drug; or a pharmaceutically acceptable salt thereof; and optionally:

(C) instructions for the simultaneous, sequential or separate administration of the compound of (A) and the benzodiazepine drug of (B) to a patient in need thereof.

In a further aspect the invention relates to a method of treatment, prevention or alleviation of an anxiety disorder of a living animal body, including a human, which method comprises the step of administering to such a living animal body in need thereof, a therapeutically effective amount of a combination of

(i) the compound exo-7-(8-H-8-aza-bicyclo[3.2.1]oct-3-yloxy)-chromen-2-one, or a pharmaceutically acceptable salt thereof; and

(ii) a benzodiazepine drug; or a pharmaceutically acceptable salt thereof.

In a further aspect the invention relates to a combination of

(i) the compound exo-7-(8-H-8-aza-bicyclo[3.2.1]oct-3-yloxy)-chromen-2-one, or a pharmaceutically acceptable salt thereof; and

(ii) a benzodiazepine drug; or a pharmaceutically acceptable salt thereof; for use as a medicament.

In a still further aspect the invention relates to a combination of

(i) the compound exo-7-(8-H-8-aza-bicyclo[3.2.1]oct-3-yloxy)-chromen-2-one, or a pharmaceutically acceptable salt thereof; and

(ii) a benzodiazepine drug; or a pharmaceutically acceptable salt thereof; for use in the treatment, prevention or alleviation of an anxiety disorder of a living animal body, including a human.

The compound exo-7-(8-H-8-aza-bicyclo[3.2.1]oct-3-yloxy)-chromen-2-one

The compound exo-7-(8-H-8-aza-bicyclo[3.2.1]oct-3-yloxy)-chromen-2-one for use according to the invention is described in WO 2006/035034 (NeuroSearch NS). The compound may be prepared by conventional methods for chemical synthesis, e.g. those described in WO 2006/035034 (Method D). In one embodiment, a salt of the as the hydrochloride salt of exo-7-(8-H-8-aza-bicyclo[3.2.1]oct-3-yloxy)-chromen-2-one.

Benzodiazepine Drugs

The benzodiazepine drugs and the pharmaceutically acceptable salts thereof for use according to the invention are known in the art and may be commercially available under different brand names, or may be obtained as described in the literature.

Examples of benzodiazepine drugs include bromazepam, chlordiazepoxide, clobazam, clonazepam, clorazepate, diazepam, flunitrazepam, lorazepam, lormetazepam, medazepam, nimetazepam, nitrazepam, nordazepam, oxazepam, and prazepam. Further examples of benzodiazepine drugs include the tricyclic benzodiazepine derivatives alprazolam, midazolam, and triazolam.

Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of formula I for use according to the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a compound of formula I for use according to the invention include, without limitation, the sodium, the potassium, the calcium, the magnesium, the zinc, the aluminium, the lithium, the choline, the lysinium, and the ammonium salt, and the like, of a compound of formula I for use according to the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the compounds for use according to the invention include examples of suitable prodrugs of the compounds modified at one or more reactive or derivatizable groups of the parent compound. Of particular interest are compounds modified at a carboxyl group, a hydroxyl group, or an amino group. Examples of suitable derivatives are esters or amides.

The compounds for use according to the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Dosage of the compound exo-7-(8-H-8-aza-bicyclo[3.2.1]oct-3-yloxy)-chromen-2-one

The dosage of the compound exo-7-(8-H-8-aza-bicyclo[3.2.1]oct-3-yloxy)-chromen-2-one is determined as the API (Active Pharmaceutical Ingredient), i.e. calculated as the free base.

In the methods according to the invention the compound exo-7-(8-H-8-aza-bicyclo[3.2.1]oct-3-yloxy)-chromen-2-one, or a pharmaceutically acceptable salt thereof is administered to a human in need thereof in a therapeutically-effective amount in the range of about 0.1-2 mg API daily.

The actual dosage of each of the active ingredients depends on the nature and severity of the disease being treated, the exact mode of administration, form of administration and is within the discretion of the physician, and may be varied by titration of the dosage to the particular circumstances of this invention to produce the desired therapeutic effect. However, a daily dosage in the range from about 0.1-2 mg API daily, such as from about 0.25-1 mg API daily, such as 0.25, 0.5 or 0.75 or 1.0 mg API daily, is suitable for therapeutic treatments.

Pharmaceutical Compositions

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

The one or more adjuvants, excipients, carriers, buffers, diluents, and/or other customary pharmaceutical auxiliaries must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragée, in powder, or in liquid form, topically such as by inhalation, by patch, enterally, such as by suppository, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

Biological Activity

The compound exo-7-(8-H-8-aza-bicyclo[3.2.1]oct-3-yloxy)-chromen-2-one may be used—alone or in combination with a benzodiazepine drug—for treating anxiety, in particular anxiety selected from the group consisting of anxiety, generalized anxiety disorder, panic disorder, panic attack, panic disorder with agoraphobia, panic disorder without agoraphobia, substance-induced anxiety disorder, phobic disorder, phobia, specific phobia, agoraphobia, agoraphobia without history of panic disorder, social anxiety disorder, social phobia, generalized social phobia, specific social phobia, obsessive-compulsive disorder, post-traumatic stress disorder, post-traumatic stress syndrome and separation anxiety disorder.

### BRIEF DESCRIPTION OF THE DRAWINGS

The present invention is further illustrated by reference to the accompanying drawing, in which:

FIGS. 1 and 2 show the effect of the compound exo-7-(8-H-8-aza-bicyclo[3.2.1]oct-3-yloxy)-chromen-2-one (Compound I or Cp I) and diazepam, respectively, in the mouse marble burying test. Compound I (0.05-0.2 mg/kg) was administered orally 60 min before testing. Diazepam (0.1-1 mg/kg) was injected via the intraperitoneal (i.p.) route 30 min before testing. *P<0.05, ***P<0.001 vs. vehicle-treated group (Student's t-test, n=8).

FIG. 3 shows that the compound exo-7-(8-H-8-aza-bicyclo[3.2.1]oct-3-yloxy)-chromen-2-one (Compound I or Cp I) augments the activity of diazepam in the mouse marble burying test. Compound I (0.05 mg/kg) was administered orally 60 min before testing. Diazepam (0.03-0.3 mg/kg) was injected via the intraperitoneal (i.p.) route 30 min before testing. Paroxetine (10 mg/kg, i.p., −30 min) served as a positive control. *P<0.05, **P<0.01 vs. vehicle-treated group (Student's t-test, n=8).

### EXAMPLES

The invention is further illustrated with reference to the following examples, which are not intended to be in any way limiting to the scope of the invention as claimed.

### Example 1

The Marble Burying Test

Mice were placed for 30 min in novel cages (one mouse per cage, 20×30 cm) in which there were 20 glass marbles (15 mm in diameter) situated in four rows of five placed on top of 5 cm of sawdust. The mean number of glass marbles buried ±S.E.M. during the 30 min test session was taken as an index of “anxiety”, i.e., the more marbles buried the more anxious the mouse-a marble was classified as buried when at least two-thirds was covered by sawdust (Broekkamp C L, Rijk H W, Joly-Gelouin D, and Lloyd K L (1986) Major tranquillizers can be distinguished from minor tranquillizers on the basis of effects on marble burying and swim-induced grooming in mice. Eur J Pharmacol 126: 223-229). The experimenter was blind to the treatments given to animals in all studies.

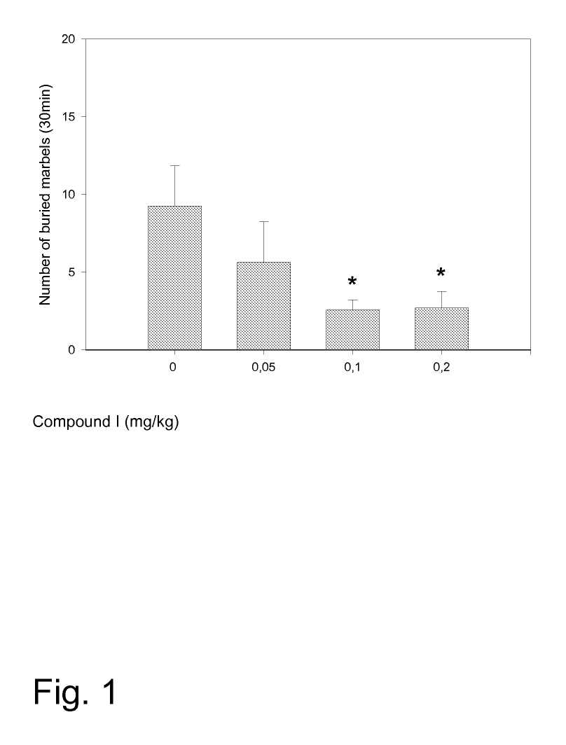

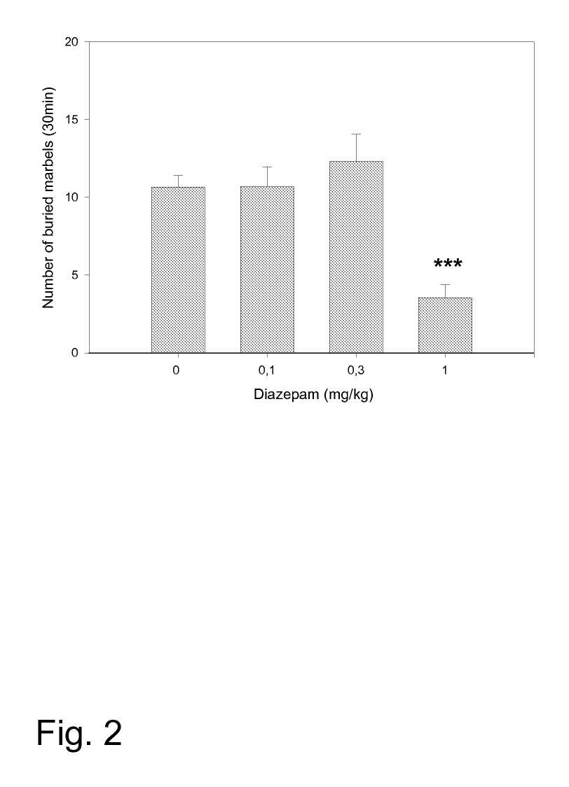

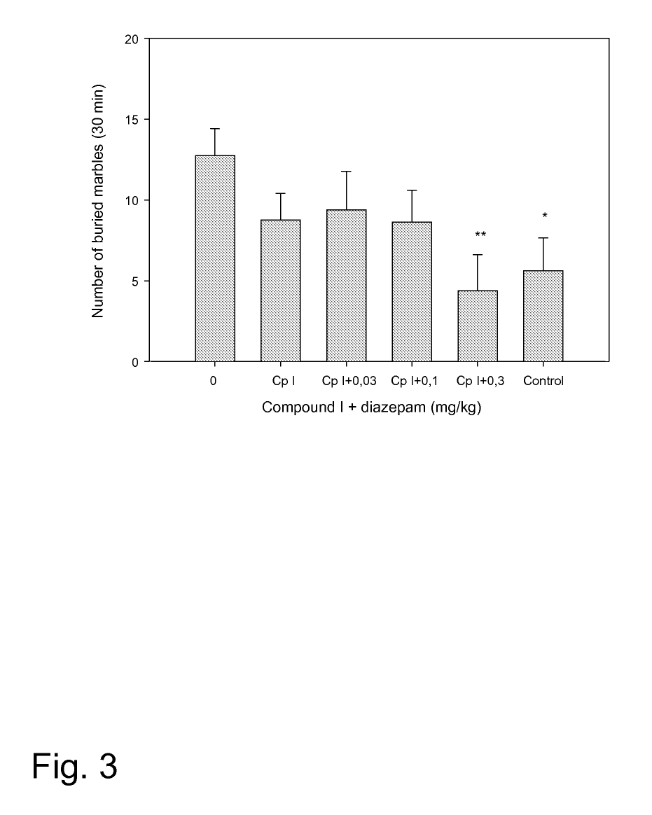

The results obtained from studying the combination of the compound exo-7-(8-H-8-aza-bicyclo[3.2.1]oct-3-yloxy)-chromen-2-one (Compound I or Cp I) alone and in combination with diazepam in the marble burying test are seen in FIGS. 1-3.

1. Method for treating an anxiety disorder comprising administering to a human a composition comprising the compound exo-7-(8-H-8-aza-bicyclo[3.2.1]oct-3-yloxy)-chromen-2-one, or a pharmaceutically acceptable salt thereof in a therapeutically-effective amount.

2. The method according to claim 1, wherein the anxiety disorder is selected from the group consisting of anxiety, generalized anxiety disorder, panic disorder, panic attack, panic disorder with agoraphobia, panic disorder without agoraphobia, substance-induced anxiety disorder, phobic disorder, phobia, specific phobia, agoraphobia, agoraphobia without history of panic disorder, social anxiety disorder, social phobia, generalized social phobia, specific social phobia, obsessive-compulsive disorder, post-traumatic stress disorder, post-traumatic stress syndrome and separation anxiety disorder.

3. The method according to claim 1, wherein the composition is administered orally, intravenously, intravascularly, intraperitoneally, subcutaneously, intramuscularly, inhalatively, topically, by patch, or by suppository.

4. The method according to claim 1, wherein the compound is administered in the range of about 0.1-2 mg API daily.

5. A pharmaceutical composition for the treatment of an anxiety disorder in a human, said composition comprising a therapeutically-effective amount of the compound exo-7-(8-H-8-aza-bicyclo[3.2.1]oct-3 -yloxy)-chromen-2-one,
or a pharmaceutically acceptable salt thereof in admixture with one or more pharmaceuticaly acceptable adjuvants, excipients, carriers and/or diluents.

6. The composition according to claim 5, wherein the anxiety disorder is selected from the group consisting of anxiety, generalized anxiety disorder, panic disorder, panic attack, panic disorder with agoraphobia, panic disorder without agoraphobia, substance-induced anxiety disorder, phobic disorder, phobia, specific phobia, agoraphobia, agoraphobia without history of panic disorder, social anxiety disorder, social phobia, generalized social phobia, specific social phobia, obsessive-compulsive disorder, post-traumatic stress disorder, post-traumatic stress syndrome and separation anxiety disorder.

7. The composition according to claim 5, wherein the composition is administered orally, intravenously, intravascularly, intraperitoneally, subcutaneously, intramuscularly, inhalatively, topically, by patch, or by suppository.

8. The composition according to claim 5, wherein the dosage of the compound of formula I is 0.1-2 mg API.

9. (canceled)

10. A pharmaceutical composition comprising a therapeutically effective amount of
(i) the compound exo-7-(8-H-8-aza-bicyclo[3.2.1]oct-3-yloxy)-chromen-2-one, or a pharmaceutically acceptable salt thereof; and
(ii) a benzodiazepine drug; or a pharmaceutically acceptable salt thereof;
together with one or more adjuvants, excipients, carriers and/or diluents.

11. The pharmaceutical composition according to claim 10, wherein the benzodiazepine drug is selected from the group consisting of bromazepam, chlordiazepoxide, clobazam, clonazepam, clorazepate, diazepam, flunitrazepam, lorazepam, lormetazepam, medazepam, nimetazepam, nitrazepam, nordazepam, oxazepam, prazepam, alprazolam, midazolam, and triazolam; and pharmaceutically acceptable salts thereof.

12. The pharmaceutical composition according to claim 11, wherein the benzodiazepine drug is diazepam; or a pharmaceutically acceptable salt thereof.

13. (canceled)

14. (canceled)

15. A kit of parts comprising at least two separate unit dosage forms (A) and (B):
(A) the compound exo-7-(8-H-8-aza-bicyclo[3.2.1]oct-3-yloxy)-chromen-2-one, or a pharmaceutically acceptable salt thereof; and
(B) a benzodiazepine drug; or a pharmaceutically acceptable salt thereof; and optionally
(C) instructions for the simultaneous, sequential or separate administration of the compound of (A) and the benzodiazepine drug of (B) to a patient in need thereof.

16. A method of treatment, prevention or alleviation of an anxiety disorder of a living animal body, including a human, which method comprises the step of administering to such a living animal body in need thereof, a therapeutically effective amount of a combination of the compound exo-7-(8-H-8-aza-bicyclo[3.2.1]oct-3-yloxy)-chromen-2-one, or a pharmaceutically acceptable salt thereof; and
(ii) a benzodiazepine drug; or a pharmaceutically acceptable salt thereof

17. A combination of
(i) the compound exo-7-(8-H-8-aza-bicyclo[3.2.1]oct-3-yloxy)-chromen-2-one, or a pharmaceutically acceptable salt thereof; and
(ii) a benzodiazepine drug; or a pharmaceutically acceptable salt thereof;
for use as a medicament.

18. A combination of
(i) the compound exo-7-(8-H-8-aza-bicyclo[3.2.1]oct-3-yloxy)-chromen-2-one, or a pharmaceutically acceptable salt thereof; and
(ii) a benzodiazepine drug; or a pharmaceutically acceptable salt thereof;
for use in the treatment, prevention or alleviation of an anxiety disorder of a living animal body, including a human.

19. The combination according to claim 18, wherein the anxiety disorder is a disorder or condition selected from the group consisting of anxiety, generalized anxiety disorder, panic disorder, panic attack, panic disorder with agoraphobia, panic disorder without agoraphobia, substance-induced anxiety disorder, phobic disorder, phobia, specific phobia, agoraphobia, agoraphobia without history of panic disorder, social anxiety disorder, social phobia, generalized social phobia, specific social phobia, obsessive-compulsive disorder, post-traumatic stress disorder, post-traumatic stress syndrome and separation anxiety disorder.
